(19) United States
(12) Patent Application Publication
(10) Pub. No.: US 2025/0268926 A1
(43) Pub. Date: Aug. 28, 2025

(54) METHOD AND COMPOSITION FOR TREATING, ALLEVIATING OR PREVENTING A DISEASE OR CONDITION CAUSED BY AIR POLLUTANT

(71) Applicant: Hong Kong Baptist University, Hong Kong (CN)

(72) Inventors: Zongwei Cai, Kowloon Tong (CN); Yuanyuan Song, Kowloon Tong (CN); Zhu Yang, Kowloon Tong (CN); Jieqing Feng, Kowloon Tong (CN)

(21) Appl. No.: 19/059,533

(22) Filed: Feb. 21, 2025

Related U.S. Application Data
(60) Provisional application No. 63/556,846, filed on Feb. 22, 2024.

(51) Int. Cl. A61K 31/685; A61K 31/198; A61K 31/7072; A61P 11/00; A61P 39/00

(52) U.S. Cl. CPC A61K 31/685; A61K 31/198; A61K 31/7072; A61P 11/00; A61P 39/00

(57) ABSTRACT

A method for treating, alleviating or preventing a disease or condition caused by an air pollutant includes administering a composition comprising an effective amount of phosphocholine, or a prodrug thereof, to a subject in need thereof. It also relates to a method and a composition for improving energy supply in lung cells exposed to an air pollutant.

### SEQUENCE LISTING

The Sequence Listing file entitled “sequencelisting” having a size of 9875 bytes and a creation date of Feb. 21, 2025, that was filed with the patent application is incorporated herein by reference in its entirety.

### TECHNICAL FIELD

The present invention relates to a method of treating, alleviating or preventing a disease or condition caused by an air pollutant, and particularly, although not exclusively, to a method of alleviating a disease or condition caused by particulate matter.

### BACKGROUND

Air pollution, particularly particulate matter (PM) with an aerodynamic diameter smaller than 2.5 μm (PM_{2.5}), is a pressing environmental risk and has been linked to approximately seven million deaths annually according to the World Health Organization (WHO, 2018). The rapid development of the economy in China escalated air pollution, responsible for about 1.85 million deaths per annum. Alarmingly for children, long-term PM_{2.5} exposure was attributed to 30.8 (95% confidence interval (CI): [28.6, 33.2]) million premature deaths in China from 2000 to 2016. Unfortunately, the mean population-weighted ambient PM_{2.5} concentration in 2019 was 35 μg/m³ across all urban areas globally, showing no substantial decline since 2000.

PM_{2.5} can result in many disorders, due to its heterogeneous composition varying continuously. The major ingredients of PM_{2.5} include both water-soluble inorganic ions (Na₊, K₊, NO₃, etc.) and organic matters (e.g., polycyclic aromatic hydrocarbons), and water-insoluble fractions (crustal elements/trace elements). Every type of compound possesses a unique impact on human health. Transition metals such as Fe, V, Cr, Ti and Ni, for example, could generate reactive oxygen species (ROS) through the Fenton reaction, and organic substances like polycyclic aromatic hydrocarbons (PAHs) and their derivatives, are thought to be highly carcinogenic and mutagenic.

Currently, antioxidants like Vitamin C and Vitamin E could help neutralize the oxidative stress caused by PM_{2.5} exposure, and medications such as corticosteroids could reduce inflammation in the respiratory systems and other affected organs. However, the effectiveness of treatment could vary from person to person, and the long-term use of corticosteroids could have side effects.

Accordingly, there remains a need for an improved method for alleviating discomfort or disease caused by PM_{2.5} exposure.

### SUMMARY OF THE INVENTION

In accordance with a first aspect of the present invention, there is provided a method for treating, alleviating or preventing a disease or condition caused by an air pollutant. The method comprises administering a composition comprising an effective amount of phosphocholine (abbreviated as Pc), or a prodrug thereof, to a subject in need thereof.

In an embodiment of the first aspect, the air pollutant comprises particulate matter.

In an embodiment of the first aspect, the particulate matter has an average aerodynamic diameter of less than 10 μm. Preferably, the particulate matter has an average aerodynamic diameter of less than 2.5 μm.

In an embodiment of the first aspect, the particulate matter comprises predominantly of inorganic ions.

In an embodiment of the first aspect, the subject suffers from or is at risk of developing energy metabolism disorders.

In an embodiment of the first aspect, the disease or disorder is associated with mitochondrial dysfunction in lung cells.

In an embodiment of the first aspect, Pc activates fatty acid oxidation to mitigate energy deficiencies caused by the air pollutant.

In an embodiment of the first aspect, the composition further comprises taurine, creatine or a combination thereof.

In an embodiment of the first aspect, the composition further comprises uridine diphosphate N-acetylglucosamine (UDP-GlcNAc), N-acetyl-aspartic acid (NAA), or a combination thereof.

In an embodiment of the first aspect, the method further comprises a step of measuring the level of Pc in a biological sample of the subject to determine if the subject has a reduced Pc level compared to an individual unexposed to the air pollutant.

In accordance with a second aspect of the present invention, there is provided a method for improving energy supply in lung cells exposed to an air pollutant, comprising contacting the lung cells with an effective amount of Pc or a prodrug thereof.

In an embodiment of the second aspect, the lung cells have mitochondrial dysfunction.

In an embodiment of the second aspect, the air pollutant comprises particulate matter.

In an embodiment of the second aspect, the particulate matter has an average aerodynamic diameter of less than 2.5 μm.

In an embodiment of the second aspect, the Pc or a prodrug thereof activates fatty acid oxidation to mitigate energy deficiencies caused by the air pollutant.

In accordance with a third aspect of the present invention, there is provided a composition for improving energy supply in lung cells exposed to an air pollutant, the composition comprises an effective amount of Pc or a prodrug thereof for activating fatty acid oxidation in lung cells.

In an embodiment of the third aspect, the composition further comprises an effective amount of taurine, creatine, UDP-GlcNAc, NAA, or a combination thereof.

In an embodiment of the third aspect, the composition comprises both Pc and UDP-GlcNAc.

In an embodiment of the third aspect, the composition is provided in a form of food supplement.

The present invention therefore provides methods for determining whether a subject has energy deficiency caused by particulate matter, particularly by measuring the level of Pc, and methods for treating, alleviating or preventing the discomfort or any conditions caused by the energy deficiency. The invention thus involves the use of Pc as a biomarker and the use of it in therapy. It would be appreciated that Pc could be developed as both a pharmaceutical composition and a food supplement depending on the application.

### BRIEF DESCRIPTION OF THE DRAWINGS

Embodiments of the present invention will now be described, by way of example, with reference to the accompanying drawings in which:

FIGS. 1A-1J illustrate the profiles of year-round PM₂ collected in 2017-2018 from Taiyuan and Guangzhou; FIG. 1A shows the Z-score of 82 main chemicals in each sample; FIG. 1B shows the seasonal and yearly averages of the absolute concentrations of the chemicals; FIG. 1C is a plot of diagnostic ratio analysis; FIG. 1D is a plot of incremental lifetime cancer risk (ILCR) analysis, in which the vertical dashed line annotates 1×10⁻⁶, U.S. Environmental Protection Agency (USEPA)'s standard for high potential carcinogenicity; FIG. 1E is a plot showing the cell viability changes with PM_{2.5} concentrations in Taiyuan, in which p values indicate the significance levels of Pearson's correlation coefficients; FIG. 1F is a plot showing the cell viability changes with PM_{2.5} concentrations in Guangzhou, in which p values indicate the significance levels of Pearson's correlation coefficients; FIG. 1G is a plot showing Dithiothreitol (DTT) activity changes with PM_{2.5} concentrations in Taiyuan; FIG. 1H is a plot showing DTT activity changes with PM_{2.5} concentrations in Guangzhou; FIG. 1I is a plot showing superoxide dismutase assay (SOD) activity changes with the DTT activity of Taiyuan samples; FIG. 1J is a plot showing SOD activity changes with the DTT activity of Guangzhou samples; note the dashed lines in FIGS. 1E to 1J are linear fits of all dots or indicated subgroups.

FIG. 2 is a schematic diagram showing selection of biomarkers through correlation analysis, in which the colored boxes indicate the fold changes of both cities against the control; significance came from t-test: *, against ctrl; #, against Taiyuan samples; ** and ##, p<0.01; *** and ###, p<0.001; and insert plots display the relative concentrations of the indicated metabolites against the total pollutant extracted from the filters, yellow and blue for Taiyuan and Guangzhou, respectively.

FIG. 3, parts A through N, illustrate the roles of selected metabolite biomarkers in the cellular response after exposure to PM_{2.5}; A is a plot showing the effects of supplying 200 μM of each biomarker on the cell viability of BEAS-2B cells exposed to 200 μg/mL PM_{2.5} samples; B is a plot showing the changes in cell viability of BEAS-2B cells exposed to 200 μg/mL PM_{2.5} samples with different concentrations of Pc, taurine, and creatine, in which data are presented as mean±SEM (n=6 per group); C to F show the effect of the selected biomarkers on oxygen consumption rate (OCR), the fold change of basal respiration, the fold change of maximal respiration, and the fold change of adenosine triphosphate (ATP) production under normal conditions after adding different biomarkers; G to J show the effect of the selected biomarkers on OCR, the fold change of basal respiration, the fold change of maximal respiration, and the fold change of ATP production under Taiyuan PM_{2.5} exposure conditions after adding different biomarkers; K to N show the effect of the selected biomarkers on OCR, the fold change of basal respiration, the fold change of maximal respiration, and the fold change of ATP production under Guangzhou PM_{2.5} exposure conditions after adding different biomarkers, in which data are presented as mean±SEM (n=3 per group). Significance against PM_{2.5}-treated samples is annotated. *, p<0.05; **, p<0.01.

FIG. 4, parts A to D illustrate the alleviating function of the biomarker Pc on PM_{2.5}-impeded energy metabolism; A is a plot showing OCR of fatty acid oxidation test; B is a plot showing the fold change in maximal respiration against the normal cells with Pc supplement.; C is a plot showing the fold change of Phosphoethanolamine/phosphocholine Phosphatase 1 (Phospho1) expression after adding Pc to the cells with or without PM_{2.5}; D is a plot showing the fold change of peroxisome proliferator-activated receptor gamma coactivator 1-alpha (PGC-1α) expression after adding Pc to the cells with or without PM_{2.5}, in which data are presented as mean±SEM (n=3 per group). Significance: *, against Ctrl; #against PM_{2.5}; * and #, p<0.05.

FIG. 5, parts A through F illustrate the beneficial effects of Pc on the cells exposed to the mixture of the environmental markers selected from the Taiyuan PM_{2.5} examples (TY1 panel); A shows the cell viability of BEAS-2B cells after exposure to various combinations from the TY1 panel; B is a plot showing ROS production after exposure to different combinations, in which data are presented as mean±SEM (n=6 per group); Significance against Ctrl is shown: *, p<0.05; **, p<0.01; C OCR of cells exposed to TY1 after Pc supply; D is a plot showing fold changes of different readouts of OCR experiments with and without Pc supplied; Significance: *, p<0.05; E is a plot showing the fold change of Phospho1 expression of the cells exposed to TY1 after Pc adding; F is a plot showing the fold change of PGC-1α expression of the cells exposed to TY1 after Pc adding; data are presented as mean±SEM (n=3 per group); Significance: *, against Ctrl; #, against TY1; * and #, p<0.05.

FIG. 6, parts A to D, illustrate the proportions of different pollutants in total PM_{2.5}; A is a chart of detected components in Taiyuan PM_{2.5}; B is a chart of detected components in Guangzhou PM_{2.5}; C shows pie charts of measured Taiyuan pollutants in their chemical classes; D shows pie charts of measured Guangzhou pollutants in their chemical classes.

FIG. 7, parts A to K illustrate the toxicity of Taiyuan and Guangzhou PM_{2.5}; A is partial least squares-discriminant analysis (PLS-DA) plot of samples from Taiyuan and Guangzhou; B is a violin plot of PM_{2.5} concentration of Taiyuan and Guangzhou PM_{2.5}; C is a violin plot of ILCR of adults of Taiyuan and Guangzhou PM_{2.5}; D is a violin plot of BaP equivalents (BaPeq) of Taiyuan and Guangzhou PM_{2.5}; E is a violin plot of PAHs and nitro-PAHs (N-PAHs) of Taiyuan and Guangzhou PM_{2.5}; F is a plot showing correlation between Taiyuan PM_{2.5} concentration and ILCR of adult; G is a plot showing correlation between Guangzhou PM_{2.5} concentration and ILCR of adult; H is a plot showing correlation between Taiyuan PM_{2.5} concentration and ILCR of adult; I is a plot showing correlation between Guangzhou PAHs and NPAHs concentrations and ILCR of adult; J is a plot showing cell viability after exposure to different concentrations of total PM_{2.5} samples collected from Taiyuan and Guangzhou; K is a plot showing comparisons of cell viability in the different regions at the same concentrations; the numbers after city abbreviations annotate the PM_{2.5} concentrations (in μg/m³) of the sampling dates, e.g., TY118 means the sample collected in Taiyuan on a day with a PM_{2.5} concentration of 118 μg/m³. Note: *, p<0.05.

FIG. 8, parts A and B illustrate the correlation analysis between cell viability after PM_{2.5} exposure and the abundant inorganic ions; A shows the cell viability varies with the concentration of sulfate; B shows the cell viability varies with the concentration of nitrate.

FIG. 9 illustrates the contributions of specific PM_{2.5} components to its oxidation potential and cellular oxidative stress that it caused.

FIG. 10, parts A to G refer to results obtained from metabolomic analysis of lung cells after PM_{2.5} exposure; A is a PLS-DA plot in the positive mode; B is a PLS-DA plot in the negative mode; C is a plot showing up-and down-regulated significant features detected by global profiling metabolomic analysis in BEAS-2B cells after exposure to Taiyuan and Guangzhou PM_{2.5}; D is a Venn diagram for the number of distinct features in both positive and negative modes; E is a volcano plot indicating changes in metabolite abundances between the control and Taiyuan PM_{2.5} group; F is a volcano plot indicating changes in metabolite abundances between the control and Guangzhou PM_{2.5} group; G is a volcano plot indicating changes in metabolite abundances between the Taiyuan and Guangzhou PM_{2.5} groups.

FIG. 10H is a heatmap of identified metabolites exposed to Taiyuan and Guangzhou PM_{2.5}.

FIG. 10I is an enrichment analysis of significant pathways altered by Taiyuan and Guangzhou PM_{2.5}.

FIG. 10J is a metabolic network of BEAS-2B cells after exposure to Taiyuan PM_{2.5}.

FIG. 10K is a metabolic network of BEAS-2B cells after exposure to Guangzhou PM_{2.5}.

FIG. 11, part A, shows correlation analysis between identified metabolites and PM_{2.5} components of Taiyuan samples; and part B shows correlation analysis between identified metabolites and PM_{2.5} components of Guangzhou samples; Note: METs: Metals; OHPs: OH-PAHs; Pur: Purines and purine derivatives; Car: Carboxylic acids and derivatives; Orgo: Organooxygen compounds; Fatty: Fatty acyls; Ind: Indoles and derivatives; Orgn: Organonitrogen compounds; OTHs: Others. The “Others” category in pollutants includes OC, EC, TC, and the cumulative amount of each type.

FIGS. 12, parts A to D, illustrate the alleviating function of the biomarker UDP-GlcNAc on PM_{2.5}-impeded energy metabolism; A is a plot showing Seahorse glucose oxidation after adding UDP-GlcNAc to the cells with or without PM_{2.5}; B is a plot showing the fold change of maximal respiration after adding UDP-GlcNAc to the cells with or without PM_{2.5}; C is a plot showing the fold change of. L-glutamine: fructose-6-phosphate amidotransferase (GFAT) expression after adding UDP-GlcNAc to the cells with or without PM_{2.5}; D is a plot showing the fold change of O-linked β-N-acetylglucosamine transferase (OGT) expression after adding UDP-GlcNAc to the cells with or without PM_{2.5} (Data are presented as mean±SEM (n=3 per group). Significance: *, against Ctrl; #against PM_{2.5}; * and #, p<0.05.

FIGS. 13, parts A to C, illustrate the cell viability of Guangzhou pollutants markers; A shows the cell viability of combined compounds (Mg²⁺, Cr, DBA, 1-NPYR, 4-OHPHE and TBT) selected in Guangzhou; B is a plot showing comparison of the average concentrations of the compounds selected from Guangzhou in both cities.; C shows the cell viability of combined compounds (TBT, 1-NPYR, 7-NBaA, 4-OHPHE and 1-OHNAP) selected in Guangzhou. Data are presented as mean±SEM (n=6 per group).

FIG. 14 is a table showing the formula and parameters used in ILCR analysis in accordance with an example of the present invention.

FIG. 15 is a table showing the effect of PM_{2.5} on metabolites in which 11 biomarkers were identified for having a significant correlation with PM_{2.5} components.

FIG. 16 is a table showing abbreviations, structures and suppliers of all the reagents and solvents used in the analysis of PM_{2.5} components of the present invention.

### DETAILED DESCRIPTION OF THE PREFERRED EMBODIMENTS

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usual and unavoidable impurities such as side products and components usually resulting from the respective preparation or method for obtaining the material such as traces of further components or solvents. “Consisting of” means that the material solely consists of, i.e. is formed by the respective element. As used herein, the forms “a”, “an”, and “the”, are intended to include the singular and plural forms unless the context clearly indicates otherwise.

The present invention in an aspect provides a method for treating, alleviating or preventing a disease or condition caused by an air pollutant. The method comprises administering a composition comprising an effective amount of Pc, or a prodrug thereof, to a subject in need thereof. The subject may be a mammal. The mammal may be a rodent such as a rat. In particular embodiments of the invention, the mammal is a human.

The disease or condition is caused by an air pollutant and is preferably caused by particulate matter (abbreviated as PM). The particulate matter may have an average aerodynamic diameter of less than 10 μm (denoted as PM₁₀), or preferably have an average aerodynamic diameter of less than 2.5 μm (denoted as PM_{2.5}). PM_{2.5} has a smaller diameter and may get into the lung tissues and blood stream of a subject more easily and therefore PM_{2.5} poses a greater risk to health of the subject.

In an embodiment, the particulate matter comprises inorganic ions, metals, organic carbons, inorganic carbons, and/or PAH. The particulate matter may comprise predominantly of inorganic ions, e.g. containing over 30%, 40%, or 50% of the total mass of the particulate matter. In an example, the PM_{2.5} contains at least 10% inorganic ions sulfate and nitrate of the total mass fraction.

Preferably, the particulate matter is PM_{2.5} and is from one or more cities with growing industrialization and urbanization. For example, in an embodiment, PM_{2.5} is from Taiyuan and/or Guangzhou in China. People living or working in these cities having severe air pollution may benefit from the present invention because the composition herein is useful to treat, alleviate or prevent the disease or condition caused by PM_{2.5}. Diseases and conditions caused by PM_{2.5} include asthma, respiratory inflammation, heart disease, lung cancer, pneumonia, chronic obstructive pulmonary disease, stroke etc. In an embodiment, the subject is exposed to the air pollutant e.g. PM_{2.5}, and may suffer from or is at risk of developing energy metabolism disorder which is associated with PM. The lung tissues or lung cells may have mitochondrial dysfunction, i.e. fails to provide sufficient energy to the cells or tissues to support the normal metabolism of the subject. This dysfunction could result in stroke, seizures, heart and kidney issues and the disease as described above.

As used herein, the terms “treating”, “treatment” and the like refer to delaying the onset of a disease, a condition and/or symptoms of the disease, effecting a partial or complete cure for the disease or condition, or reducing the severity of the disease or condition. The term “alleviating” refers to reduction or elimination of one or more symptoms of a pathological state or disease, and/or reducing the rate or delaying the onset or severity of one or more symptoms of a pathological state or disease, and/or preventing the pathological state or disease.

The present method includes a step of administering a composition having an effective amount of Pc or a prodrug thereof for treating, alleviating or preventing the aforesaid disease or condition. The Pc of the present invention is proven to be effective in rejuvenating lung cells which were exposed to PM_{2.5}. Pc could activate fatty acid oxidation to mitigate energy deficiencies caused by the air pollutant. It functions by switching the normal glucose-dependent energy metabolism to a fatty acid-dependent metabolism.

As used herein, the term “effective amount” refers to the amount of the Pc or its prodrug sufficient to effect beneficial or desired results. An effective amount can be administered in one or more administrations, applications or dosages and is not intended to be limited to a particular formulation or administration route. In an embodiment, Pc is administered to the subject at a concentration of about 10 μM to about 1000 μM, about 50 μM to about 500 μM, about 100 μM to 200 μM, or preferably 200 μM.

The term “prodrug” as used herein refers to a compound that could be transformed in vivo into Pc after administration. The transformation could be done via a chemical or physiological process (e.g., enzymatic processes and metabolic hydrolysis). Preferably, the prodrug is inactive when administered to a subject, and is transformed into its active form when it is delivered to the target site for exerting a therapeutic effect. The prodrug compound often offers advantages of solubility, tissue compatibility or delayed release in a subject.

The composition comprising Pc or its prodrug may be administered by any administration way including inhalative, oral, or intravenous administration. In an embodiment, the composition is formulated for oral or inhalative administration. The composition may further include one or more other active ingredients to treat, alleviate or prevent the related disease or condition. It would be appreciated that the composition may further include an excipient, in particular a pharmaceutically acceptable carrier, and it is formulated for the designed administration route.

In one embodiment, the composition further comprises taurine, creatine, UDP-GlcNAc, NAA, or a combination thereof. The inventors found that taurine and creatine have protective effect against PM_{2.5} damages, and that UDP-GlcNAc and NAA can improve the basal respiration, maximal respiration and ATP production in cells. In a preferred embodiment, the composition comprises an effective amount of both Pc and UDP-GlcNAc for treating or alleviating symptoms associated with the PM_{2.5} damages.

In a preferred embodiment, the method further comprises a step of measuring the level of Pc in a biological sample of the subject to determine if the subject has a reduced Pc level compared to an individual unexposed to the air pollutant. The measurement step could be performed before the administration step.

The biological sample used may be a biological fluid obtained from a subject, preferably a mammal such as a human, or may be obtained from cells or a cell population. In an embodiment, the sample may be a biological fluid selected from the group consisting of serum, blood, transcellular fluid, interstitial fluid, saliva, and exudates. In an embodiment, the sample is serum, or cells particularly lung cells obtained from a human. In another embodiment where the subject is a rodent, the sample may be cells or tissues from lung or liver.

The present invention in a further aspect provides a method of determining the degree of exposure of a subject's lungs to an air pollutant preferably to PM_{2.5}. The method comprises a step of obtaining a biological sample from the subject, and a step of measuring the amount or level of Pc in the biological sample and comparing the measured value to the amount or level of Pc in an individual unexposed to the air pollutant. In an embodiment, if the subject has a reduced level of Pc, it is determined that the subject is or has a likelihood of suffering from energy deficiencies caused by the air pollutant. The subject might also have mitochondrial dysfunction, energy metabolism disorder caused by the air pollutant, and/or any disease or condition as described above. In a preferred embodiment, the method further comprises a step of administering an effective amount of Pc to the subject to treat or alleviate the disease or condition.

The present invention in another aspect further provides a method for improving energy supply in lung cells exposed to an air pollutant, comprising contacting the lung cells with an effective amount of Pc or a prodrug thereof. The lung cells exposed to the air pollutant as described above, in particular particulate matter, could have a substantial decrease in energy metabolism, or specifically mitochondrial dysfunction, resulting in insufficient energy generation. In order to improve the energy supply, the aforesaid composition having the Pc or its prodrug could be provided to be in contact with the lung cells so as to activate the fatty acid-dependent metabolism.

In a further aspect, the present invention also provides a composition for improving energy supply in lung cells exposed to an air pollutant. The composition comprises an effective amount of Pc or its prodrug thereof for activating fatty acid oxidation in lung cells.

In a preferred embodiment, the composition further comprises an effective amount of taurine, creatine, UDP-GlcNAc, NAA, or a combination thereof. More preferably, it comprises both Pc and UDP-GlcNAc.

It would be appreciated that the composition herein could be provided in a form of food supplement or a pharmaceutical composition depending on the application. In an embodiment where it is provided as a supplement, people living in cities having a high degree of air pollution with particulate matter could take it for preventing the development of the disease or condition caused by PM_{2.5}, or for alleviating any discomfort caused by it.

### EXAMPLES

### Example 1

### PM_{2.5} Assessments in Taiyuan and Guangzhou

The inventors selected Taiyuan and Guangzhou for PM_{2.5} sample collection. The former suffered from the PM_{2.5} pollution caused by large coal production and consumption in the region, whereas the latter from the differing pollution patterns by growing industrialization and urbanization. The inventors conducted a year-long PM sampling in Taiyuan and Guangzhou, from May 2017 to April 2018, to decipher the primary constituents of all PM_{2.5} samples in these regions. The focus was on identifying the carbon contents, inorganic ions, metals, PAHs, and PAH derivatives within the samples. Organic carbon (OC) and elemental carbon (EC) comprise a significant portion of PM. Inorganic ions are also the most abundant soluble ingredients in PM_{2.5} composition in various regions, including Taiyuan. Despite being present in only trace amounts, metals and PAHs (including their derivatives) play a pivotal role in the toxicity of PM_{2.5}. With reference to Table 1, the inventors analyzed a comprehensive list of components including 8 inorganic ions, 14 metals, 17 PAHs, 18 N-PAHs, 11 hydroxy-PAHs (OH-PAHs), and 14 polycyclic aromatic sulfur heterocycles (PASHs).

TABLE 1
Detected species and specific components

| Species | Components |
|---|---|
| Carbon (2) | Organic carbon (OC), and Elemental carbon (EC) |
| Oons (8) | Sodium ion (Na⁺), Ammonium ion (NH⁴⁺), potassium ion (K⁺), |
| | Magnesium ion (Mg²⁺), Calcium ion (Ca²⁺), Chloride ion (Cl⁻), Sulfate |
| | (SO₄²⁻), and Nitrate (NO₃⁻) |
| Metals (14) | Titanium (Ti), Vanadium (V), Chromium (Cr), Manganese (Mn), Ferrum |
| | (Fe), Cobalt (Co), Nickel (Ni), Zinc (Zn), Arsenic (As), Selenium (Se), |
| | Strontium (Sr), Molybdenum (Mo), Cadmium (Cd), and Lead (Pb). |
| PAHs (17) | Naphthalene (NAP), Acenaphthylene (ACY), Biphenyl (BP), |
| | Acenaphthene (ACE), Fluorene (FLU), Anthracene (ANT), Phenanthrene |
| | (PHE), Fluoranthene (FLT), Pyrene (PYR), Chrysene (CHR), |
| | Benz(a)anthracene (BaA), Benzo(b)fluoranthene (BbF), |
| | Benzo(k)fluoranthene (BkF), Benzo(a)pyrene (BaP), Indeo(1,2,3- |
| | cd)pyrene (IcdP), Benzo(g,h,i)perylene (BghiP), and |
| | Dibenz(a,h)anthracene (DBA). |
| N-PAHs (18) | 1-Nitronaphthalene (1-NNAP), 2-Nitronaphthalene (2-NNAP), 2- |
| | Nitrobiphenyl (2-NBP), 3-Nitrobiphenyl (3-NBP), 4-Nitrobiphenyl (4- |
| | NBP), 1,3-Dinitronaphthalene (1,3-DNNAP), 3-Nitrodibenzofuran (3- |
| | NDBF), 2-Nitrofluorene (2-NFLU), 9-Nitroanthracene (9-NANT), 1,8- |
| | Dinitronaphthalene (1,8-DNNAP), 9-Nitrophenanthrene (9-NPHE), 3- |
| | Nitrophenanthrene (3-NPHE), 2-Nitroanthracene (2-NANT), 2- |
| | Nitrofluoranthene (2-NFLT), 1-Nitropyrene (1-NPYR), 2-Nitropyrene (2- |
| | NPYR), 7-Nitrobenz[a]anthracene (7-NBaA), and 6-Nitrochrysene (6- |
| | NC). |
| OH-PAHs (11) | 1-Hydroxynaphthalene (1-OHNAP), 2-Hydroxynaphthalene (2-OHNAP), |
| | 2-Hydroxybiphenyl (2-OHBP), 3-Hydroxybiphenyl (3-OHBP), 4- |
| | Hydroxybiphenyl (4-OHBP), 2-Hydroxyfluorene (2-OHFLU), 3- |
| | Hydroxyfluorene (3-OHFLU), 4-Hydroxyphenanthrene (4-OHPHE), 1- |
| | Hydroxyphenanthrene (1-OHPHE), 3-Hydroxyphenanthrene (3-OHPHE), |
| | and 2-Hydroxyphenanthrene (2-OHPHE). |
| PASHs (14) | Dibenzothiophene (DBT), 4-Methyldibenzothiophene (4-MDBT), 2- |
| | Methyldibenzothiophene (2-MDBT), 3-Methyldibenzothiophene (3- |
| | MDBT), 1-Methyldibenzothiophene (1-MDBT), 4,6- |
| | Dimethyldibenzothiophene (4,6-DMDBT), 2,8- |
| | Dimethyldibenzothiophene (2,8-DMDBT), 1,4- |
| | Dimethyldibenzothiophene (1,4-DMDBT), Tribenzothiophene (TBT), 2- |
| | Nitrodibenzothiophene (2-NDBT), Benzo[b]naphtho[1,2-d]thiophene |
| | (1,2-d), Benzo[b]naphtho[2,3-d]thiophene (2,3-d), 7- |
| | Methylbenzo[b]naphtho[2,3-d]thiophene (7-M(2,3-d)), and 2,8- |
| | Dinitrodibenzothiophene (2,8-DNDBT). |

Alongside OC and EC, the inventors successfully detected a total of 84 components in PM_{2.5} across both cities. In addition, they conducted untargeted metabolomics in BEAS-2B cells exposed to these air pollutants to compare differential alterations in metabolites caused by various PM_{2.5} constituents. The results not only pinpointed the components dominantly contributing to PM_{2.5} cytotoxicity but also found the functional metabolites that alleviated PM_{2.5}-induced dysfunction.

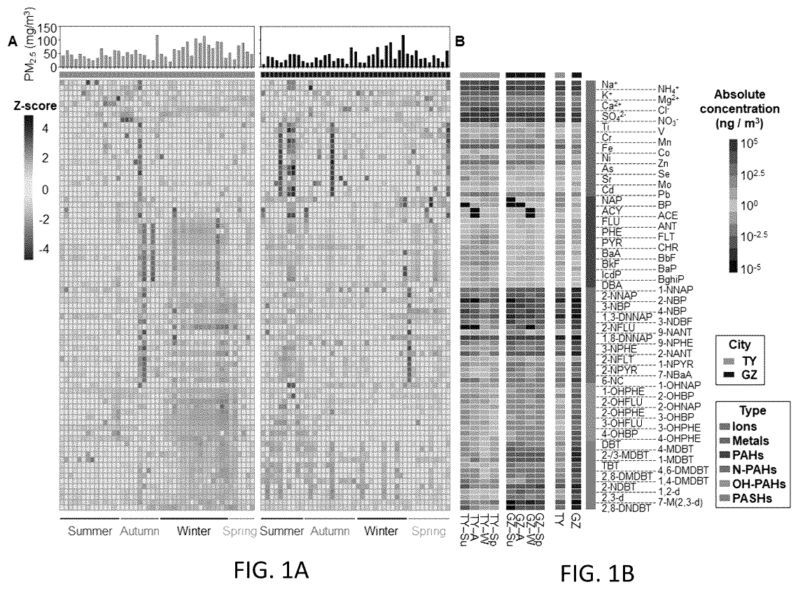

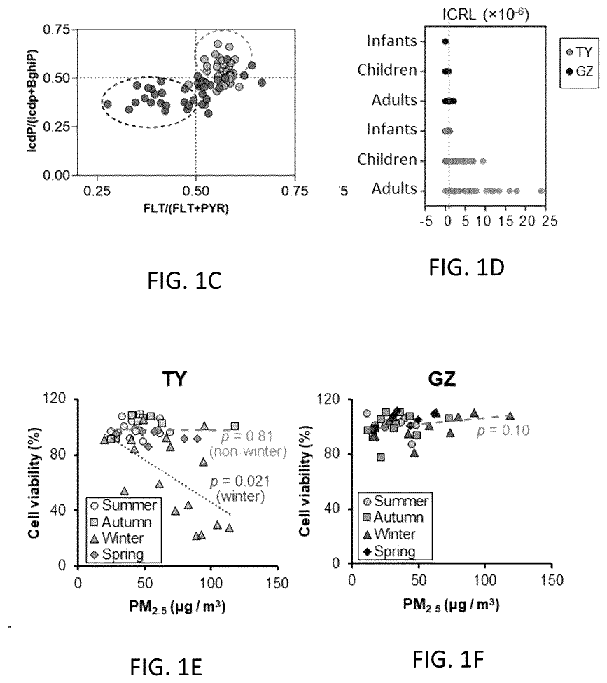

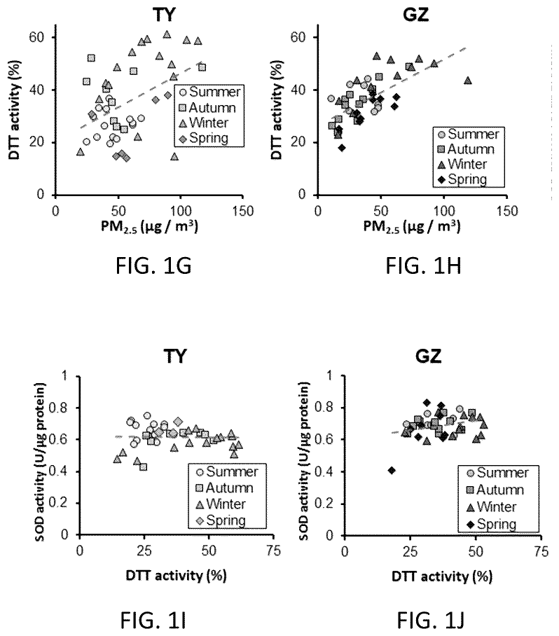

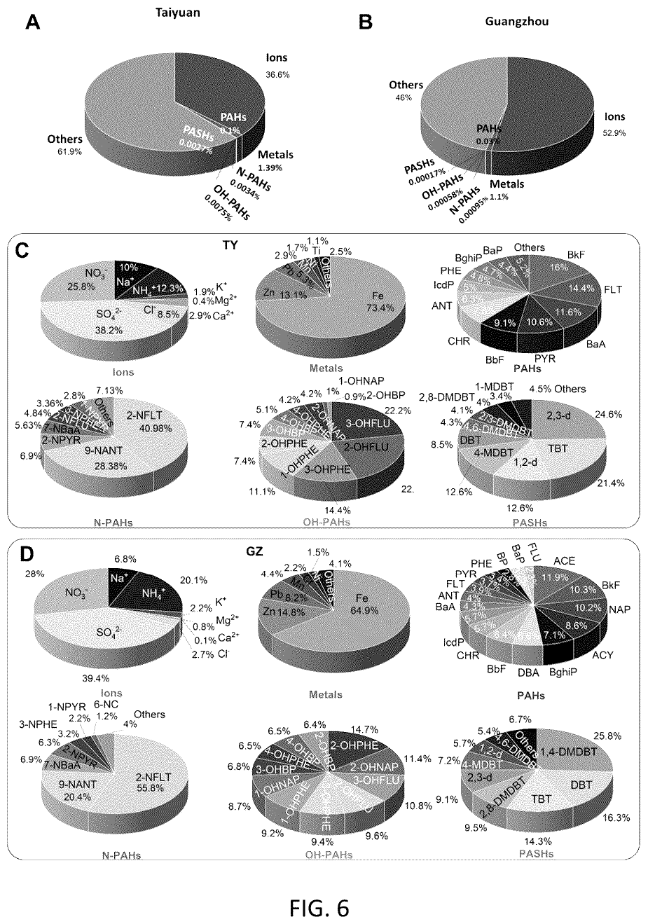

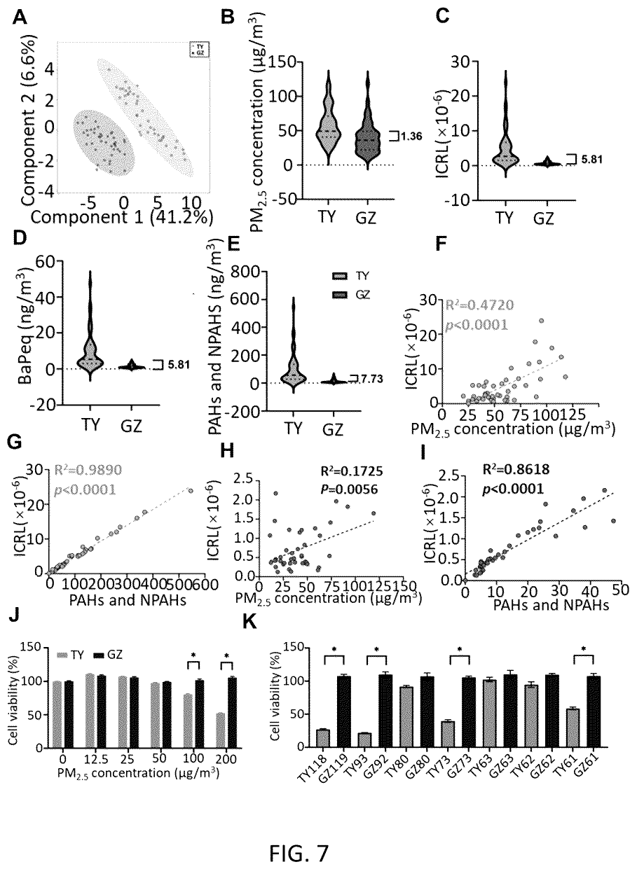

During the sampling period, the average PM_{2.5} levels were 57.7 μg/m³ and 39.9 μg/m³ in Taiyuan and Guangzhou, respectively, exceeding the recommended limits of both China (35 μg/m³) and WHO (10 μg/m³). The inventors identified and quantified 82 chemicals in Taiyuan and Guangzhou PM_{2.5} samples, spanning ions, metals, PAHs, N-PAHs, OH-PAHs, and PASHs. In general, Taiyuan had higher levels in terms of either total or individual component concentrations, with more pronounced seasonal fluctuation (FIGS. 1A and 1B). The mass proportions of individual pollutants, which were mainly determined by their types, varied dramatically from each other, showing nearly 10 orders of magnitude differences in concentrations in both cities (FIG. 1B). Though contaminate concentrations varied with date and season changes, the overall chemical class profiles were similar between the two cities: inorganic ions were the most abundant class (4.5-54.2×10³ ng/m³), followed by metals (2.8×10⁻³-3.1×10³ ng/m³) and PAHs (0.06-109.0 ng/m³), while PAHs derivatives, including N-PAHs, OH-PAHs and PASHs were the lowest (2.8×10⁻⁵-19.9 ng/m³) (FIGS. 1A, 1B, 6A and 6B). Among all analyzed chemicals, the inorganic ions sulfate (SO₄²⁻) and nitrate (NO₃⁻) emerged as the most abundant constituents, comprising one-tenth or even more of the total mass fraction in both cities (FIGS. 6A-6D). Individually, relative abundances of inorganic components were similar in both cities, whereas, for PAHs and their derivatives, a larger difference in the relative proportions of these components was observed between the two cities (FIG. 6C and FIG. 6D). For example, FLT and BaA accounted for 14.4% and 11.6% of Taiyuan PAHs, respectively, but these two numbers were 3.9% and 4.3% in Guangzhou; the top two PASHs in Taiyuan were 2,3-d (24.6%) and TBT (21.4%), which accounted for nearly one-half of PASHs, while the top two in Guangzhou were 1,4-DMDBT (25.8%) and DBT (16.3%), together sharing a similar proportion in this city (FIG. 1B). PLS-DA using these detected components distinctly separated Taiyuan samples from Guangzhou ones, highlighting their disparities in composition (FIG. 7A).

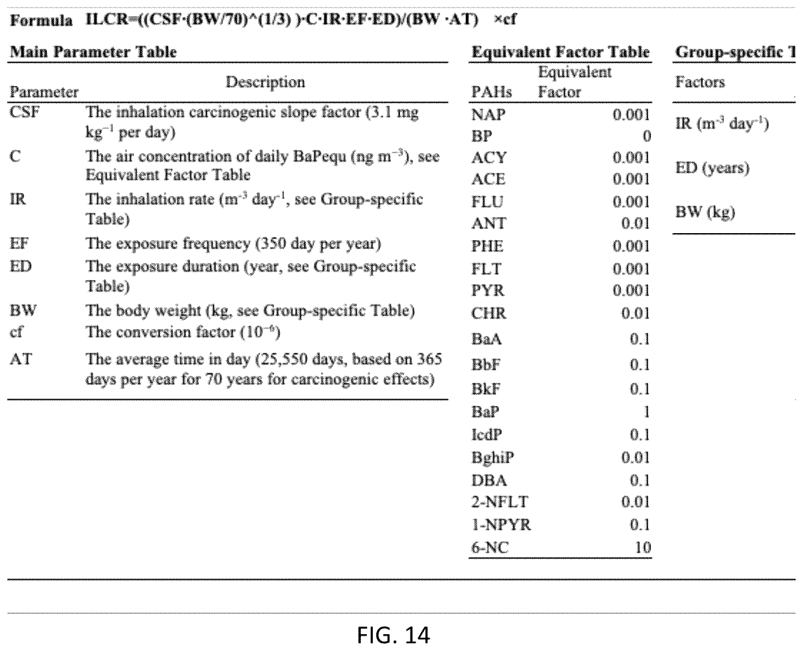

The variation implies differences in sources and potential health risks. Source apportionment, health risk, and cytotoxicity analyses were conducted to better understand these. The diagnostic ratio displayed that most (72%) of the Taiyuan PM_{2.5} samples had both ratios of FLT/(FLT+PYR) and IcdP/(IcdP+BghiP) higher than 0.5, indicating that coal combustion was one of the major sources of PM_{2.5} in Taiyuan (FIG. 1C). In Guangzhou, these two ratios in half of the sampling dates were smaller than 0.5, suggesting that traffic emission was the predominant PM_{2.5} source in this city. Then, the inventors employed ILCR models from the USEPA to assess inhalation risk based on the detected PAHs and N-PAHs and the average demographic parameters. FIG. 14 shows the formula of ILCR model and the parameters used for the ILCR analysis. Unsurprisingly, the average ILCR levels in both cities elevated with the increase in age (FIG. 1D). In Taiyuan, most sampling days (37 out of 44 for adults, 22 out of 44 for children, and 1 out of 44 for infants) had ILCRs higher than 1×10⁻⁶, USEPA's standard for high potential carcinogenicity. For the pollution levels of some days (7 out of 44), the ILCR values even exceeded 1×10⁻⁵, ten times higher than the line. In Guangzhou, on the other hand, only in 27.3% of sampling days (12 out of 44), inhalation ICRL for adults exceeded the threshold. The regional difference in carcinogenic risks caused by PAHs and N-PAHs was much larger than that in PM_{2.5} levels (FIGS. 7B-I), indicating that the toxicity of PM_{2.5} was attributed not only to the total PM_{2.5} concentrations but also to the composition of the pollutants.

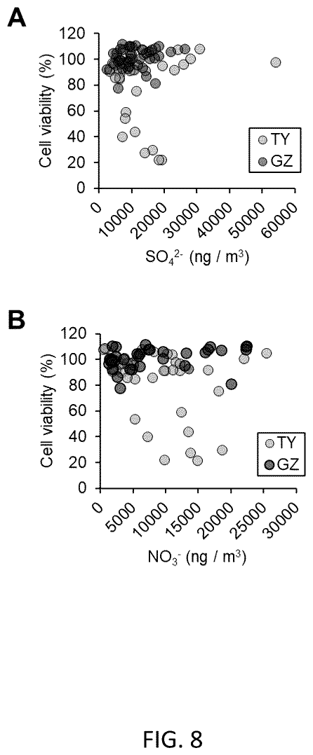

Cytotoxicity assays on BEAS-2B cells exposed to the PM_{2.5} samples collected in both cities yielded varied results. Challenging the cells with the Taiyuan PM_{2.5} samples collected in summer, autumn, and spring resulted in the cell viability from 86% to 109%, similar to the range of cell viability (78% to 112%) after exposure to the Guangzhou PM_{2.5} samples (FIGS. 1E and 1F). A negative correlation was observed between cell viability and the amounts of winter PM_{2.5} samples from Taiyuan (FIG. 1E). Interestingly, the PM_{2.5} in Taiyuan from certain dates showed much stronger toxicity than the samples in Guangzhou of the same concentration in terms of cell viability (FIGS. 1E, 7J and 7K). This result was consistent with the previous report that the same concentrations of PM_{2.5} in Taiyuan and Guangzhou produced various degrees of oxidative stress and inflammatory responses in three different lung cell lines. Despite sulfate and nitrate making up significant portions of the mass fraction, they showed no correlation with cell viability following PM_{2.5} exposure (FIG. 8). The findings indicated that the water-soluble fraction of PM_{2.5}, predominantly comprised of inorganic ions, exhibited significantly lower toxicity compared to the water-insoluble (or organic-soluble) fraction of PM_{2.5}. This underscores the notion that PM_{2.5} toxicity cannot be solely attributed to concentration.

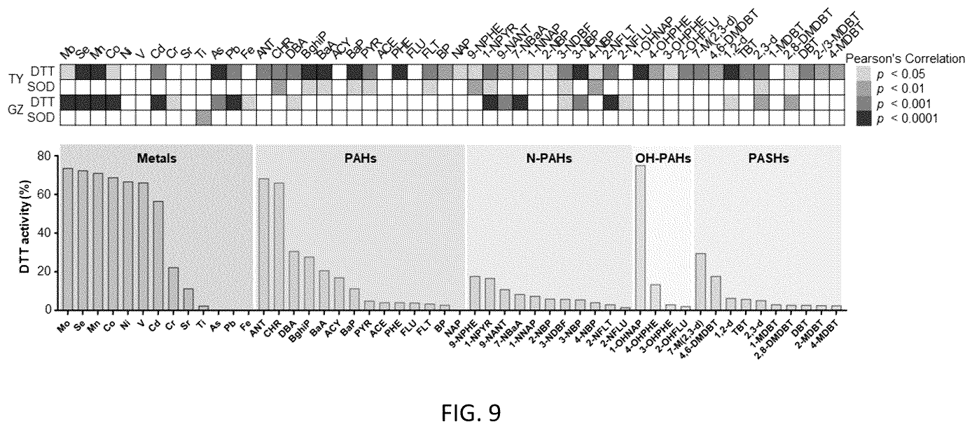

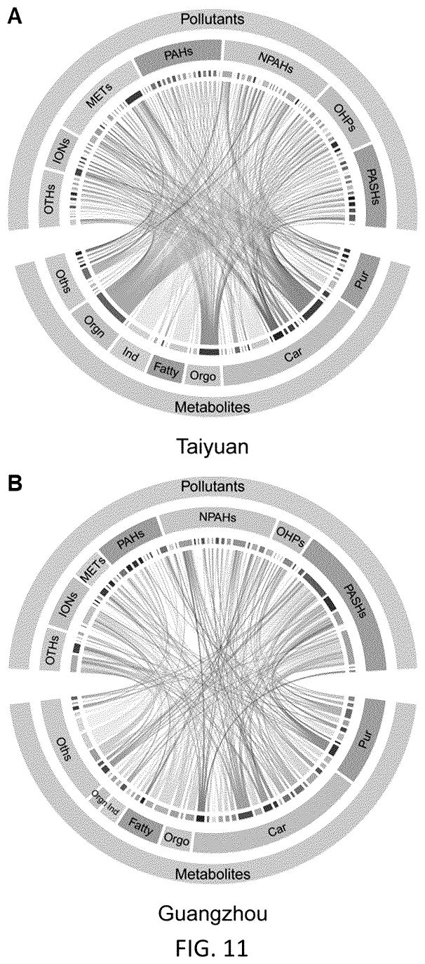

The DTT assay, a widely used non-cellular method, was employed to assess the oxidative potential of pollutants. The results indicated a positive correlation between PM_{2.5} concentrations and their DTT activities in both Taiyuan and Guangzhou (FIGS. 1G and 1H), suggesting that high levels of PM_{2.5} have high oxidative potential. However, the oxidative stress caused by the PM_{2.5} exposure in BEAS-2B cells, as determined with SOD, was not correlated with the oxidative potential of the pollutant samples (FIGS. 11 and 1J), indicating that the toxicity of PM_{2.5} was primarily dependent on the bioavailability of the components and their interactions with the biological systems. It has been documented that the most toxic metals bound to PM_{2.5} are mainly distributed in bioavailable fractions, which are water-or acid-soluble. Once solved, these metals are capable of producing ROS through Fenton-like reactions, leading to cytotoxic effects. The present DTT assay conducted on specific PM_{2.5} components confirmed that many of these transition metals exhibited a high level of oxidative potential (FIG. 9). Moreover, their concentrations in PM_{2.5} were strongly correlated with the oxidative potential of PM_{2.5}, particularly in the Taiyuan samples. However, these metals demonstrated weaker correction with the oxidative stress caused by PM_{2.5} exposure compared to organic components (FIG. 9). Taken together, these results indicated that PM_{2.5} toxicological studies should not only focus on the overall concentration but also specific compositions of PM_{2.5}.

### Example 2

### Metabolomic Analysis of Cellular Toxicity

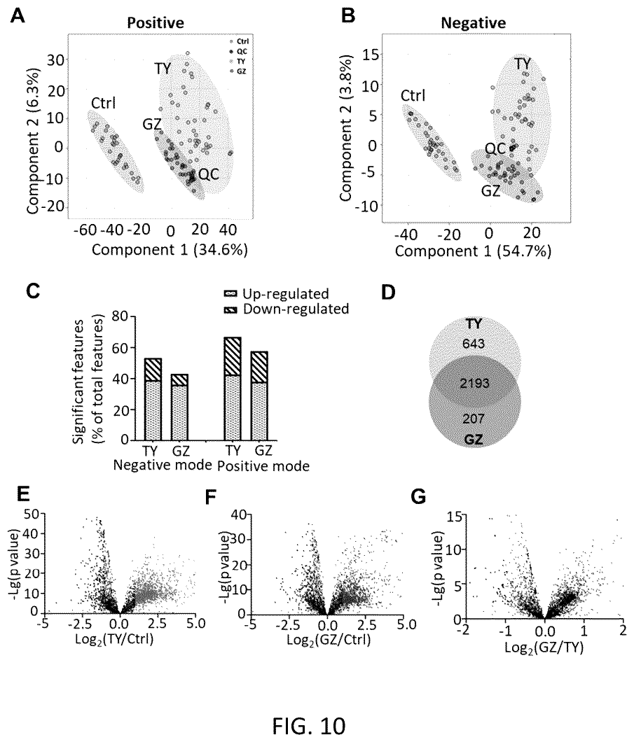

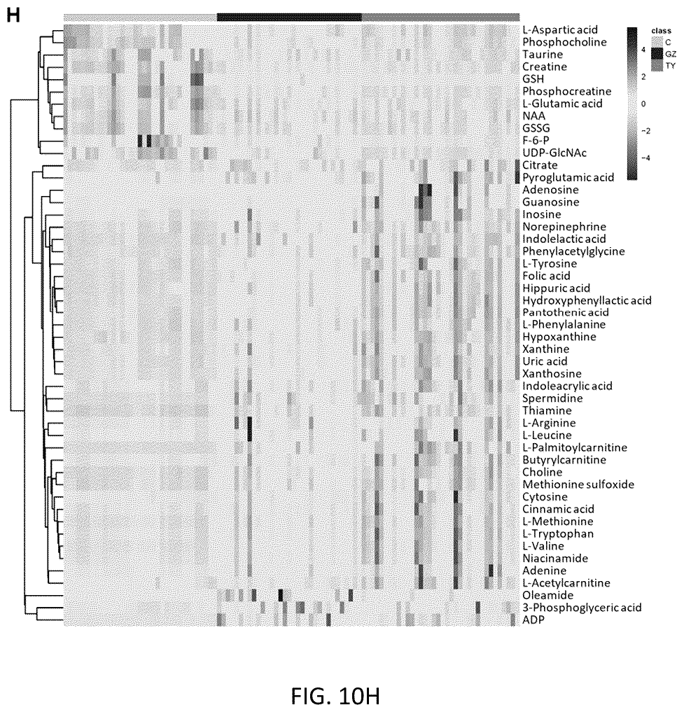

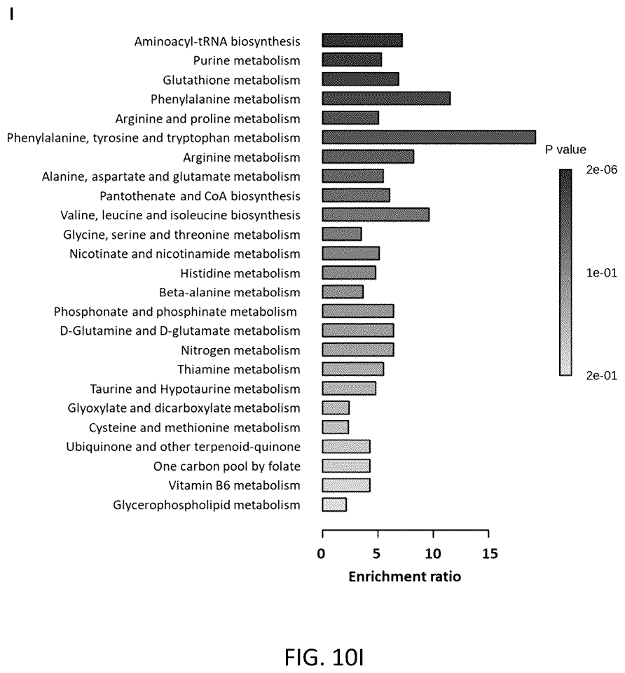

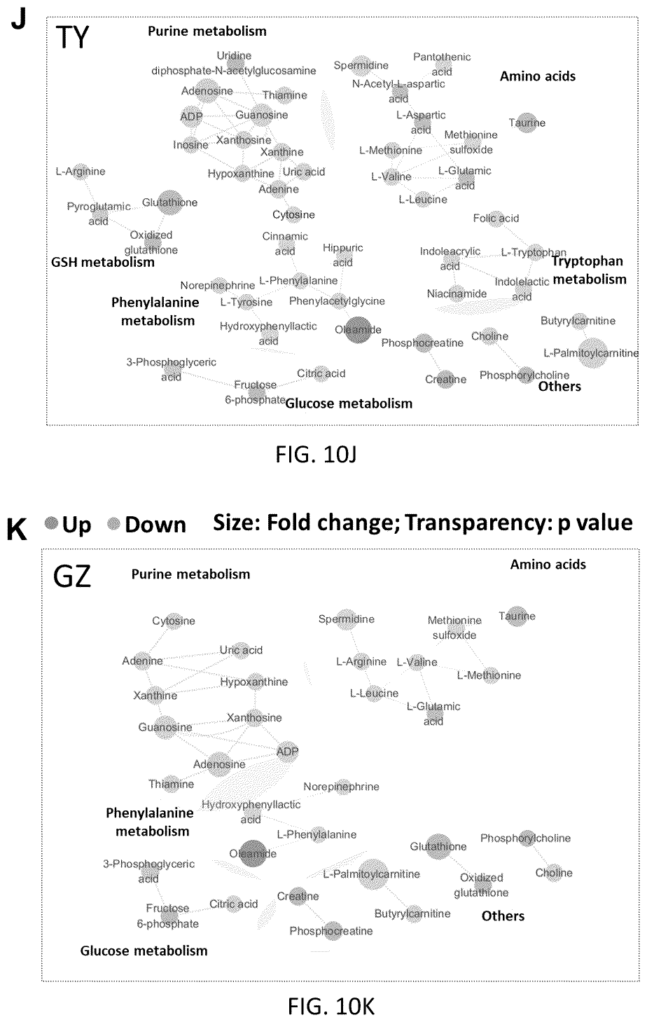

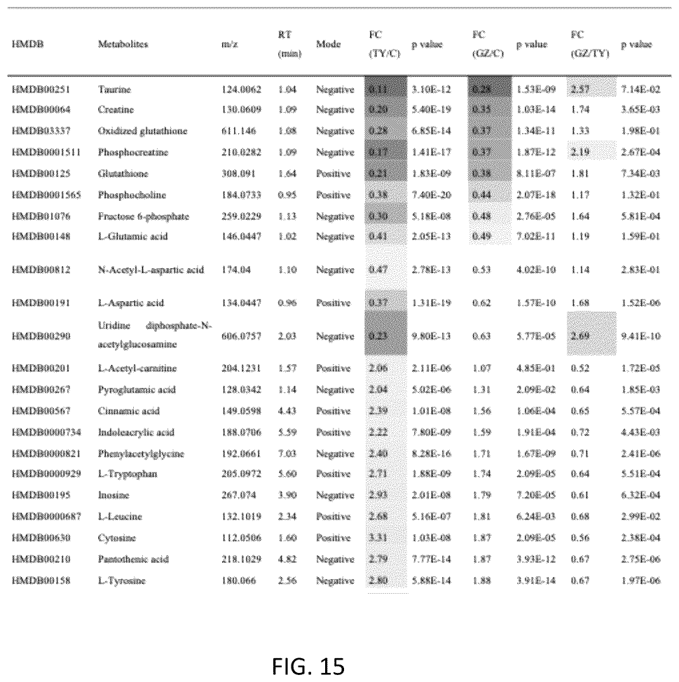

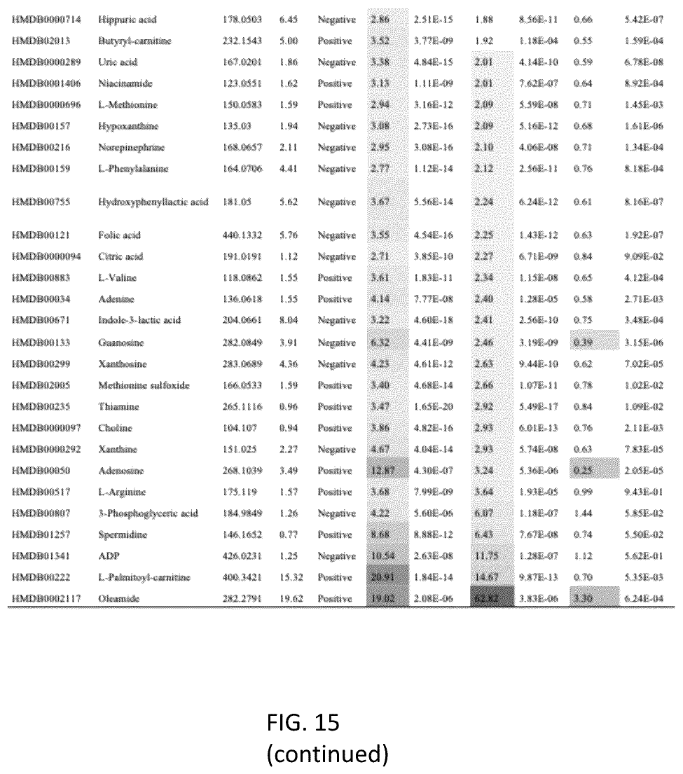

The inventors conducted global metabolomic profiling in BEAS-2B cells, upon exposure to the PM_{2.5} samples from Taiyuan and Guangzhou. BEAS-2B cells, immortalized human bronchial epithelial cells known to respond similarly to primary lung cells, are widely used in research on PM_{2.5} toxicities. The PLS-DA plots (FIGS. 10A and 10B) demonstrated the distinct metabolic disturbances caused by Taiyuan and Guangzhou samples, while PM_{2.5} from each city induced specific changes in the cells. Although not identical, both cities' PM_{2.5} induced similar changes in the metabolome, with Taiyuan's PM_{2.5} causing more pronounced metabolic disruption (FIGS. 10C to 10G). In total, Taiyuan and Guangzhou samples significantly altered 49 and 33 metabolites, respectively (FIG. 10H and FIG. 15). These metabolites changed the same ways after exposure to PM_{2.5} from both cities, and Taiyuan pollutants resulted in larger alterations in general. The changes of all the metabolites varied with sampling dates (FIG. 10H), suggesting that the disturbance of cellular metabolism might be associated with both the dose and composition of PM_{2.5}. The enrichment analysis demonstrated that pathways related to oxidative stress (purine metabolism and glutathione metabolism) and amino acids metabolism (e.g. phenylalanine metabolism, arginine and proline metabolism, and phenylalanine, tyrosine and tryptophan metabolism) were significant (FIGS. 10I-10K).

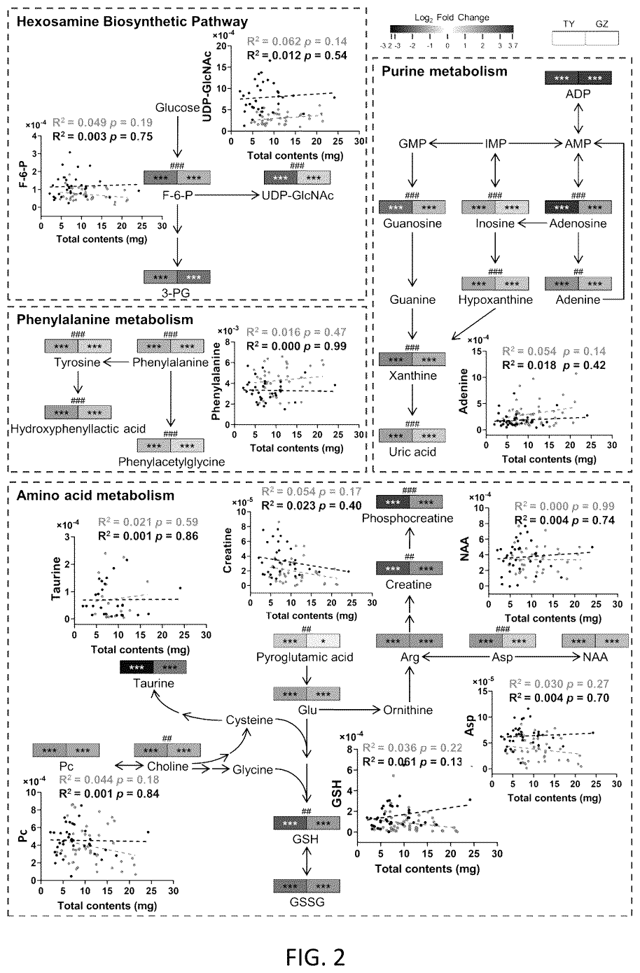

Correlation analysis identified 11 biomarkers significantly (p<0.05) correlated with a lot (≥10) of PM_{2.5} components (FIGS. 2, 11A, 11B, 15), including creatine, glutathione (GSH), Pc, fructose 6-phosphate (F-6-P), glutamic acid (Glu), aspartic acid (Asp), UDP-GlcNAc, Phenylalanine (Phe), adenine, oleamide, taurine and NAA. Notably, none of these 11 cellular metabolites showed a correlation with the total amount of PM_{2.5} (FIG. 2), highlighting the importance of PM_{2.5} composition in toxicity. Most (8 out of 11) of these metabolites were reduced after PM_{2.5} treatment, although the PM_{2.5} exposure caused the increases of 38 metabolites and the decreases of only 11 ones (FIG. 15).

### Example 3

### Alleviating Effects of the Functional Biomarkers

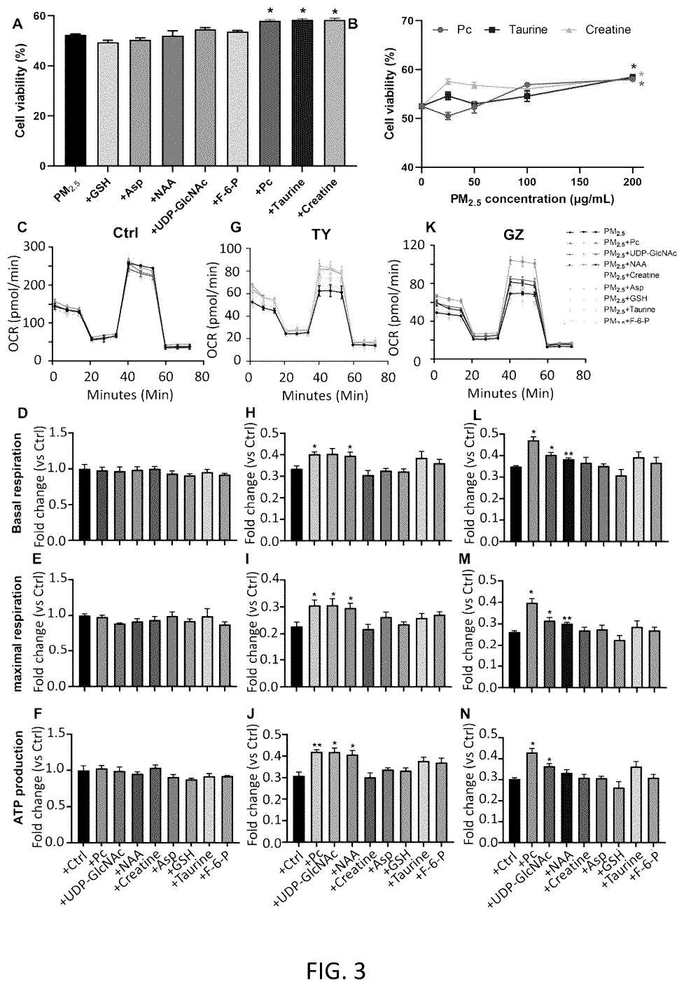

The inventors then investigated the effects of supplying these metabolites that were shortfall post PM_{2.5} exposure. Pc, taurine, and creatine could improve the cell viability reduced by PM_{2.5} exposure, suggesting that these compounds have protective effects against PM_{2.5} damage (FIGS. 3A and 3B). Mitochondria in human lung cells are specific targets of PM_{2.5} cytotoxicity and energy metabolism is important on cell survival after PM_{2.5} invasion. The inventors then evaluated the impacts of these metabolites on cellular OCR.

Under normal growth conditions, none of these metabolites influence the efficiency of mitochondrial respiration (FIGS. 3C-F). However, after exposure to PM_{2.5} from both Taiyuan (FIGS. 3G-J) and Guangzhou (FIGS. 3K-N), administration of Pc, UDP-GlcNAc and NAA could significantly increase the basal respiration, maximal respiration and adenosine triphosphate (ATP) production in cells, indicating the alleviating roles of these metabolites in PM_{2.5}-impeded energy metabolism.

These results suggested potential mitigation strategies for Pc, which could significantly reduce the cellular toxicity and mitochondrial interference caused by PM_{2.5} exposure. The inventors further explored the mechanistic role of Pc in ameliorating energy metabolism disorder after PM_{2.5} damage.

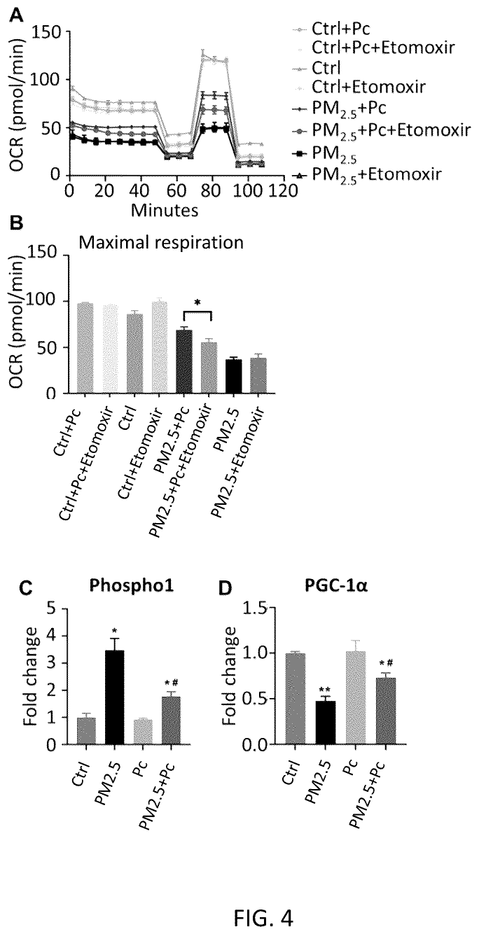

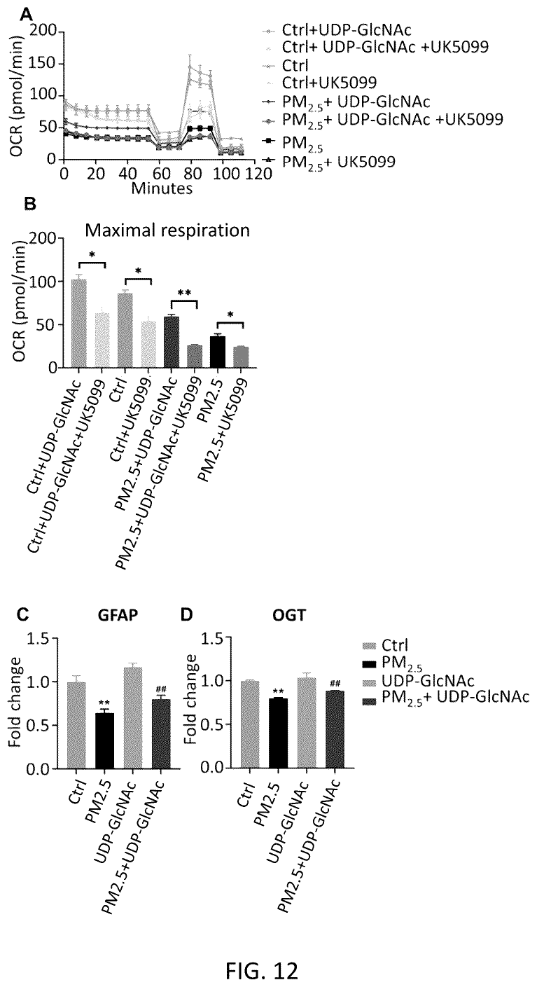

Despite the suppressive effects of PM_{2.5}, UK5099, the inhibitor of glucose oxidation, remarkably reduced the maximal respiration of cells (FIGS. 12A and 12B), whereas etomoxir, the inhibitor of fatty acids oxidation, did not change that (FIGS. 4A and 4B). These results suggested that lung cells mainly use glucose but not fatty acids for energy supply either under normal circumstances or in response to PM_{2.5} toxicity.

Adding UDP-GlcNAc increased (1.6 fold) cells' glucose oxidation in the cultures with and without PM_{2.5}, at both of which were suppressed by UK5099 (FIGS. 12A and 12B). These data indicated the beneficial effects of elevated UDP-GlcNAc on glucose metabolism, especially under PM_{2.5} conditions which might impair the energy supply from glucose.

Intriguingly, the administration of Pc significantly increased the maximal respiration only in the PM_{2.5}-exposed lung cells, in which etomoxir also functioned (FIGS. 4A and 4B). These data demonstrated that applying Pc could reprogram cellular metabolism to fatty acid oxidation to meet the shortfall in energy supply during the PM_{2.5} challenge.

The expression of the related genes further confirmed these responses GFAT is the rate-limiting enzyme in the hexosamine biosynthetic pathway and initializes the metabolism to UDP-GlcNAc. After exposure to PM_{2.5}, GFAT was downregulated significantly, while adding UDP-GlcNAc to the cell medium interacted with the decline of GFAT (FIG. 12C). OGT catalyzes the post-translational modification of serine/threonine protein residues by O-linked β-N-acetylglucosamine (O-GlcNAc). The activity of OGT is sensitive to its substrate UDP-GlcNAc. OGT expression was also reduced after PM_{2.5} exposure and partially rescued by adding UDP-GlcNAc (FIG. 12D). Phospho1 is the primary enzyme controlling Pc content. In the normal cells, additional Pc showed no effects on Phosoho1 expression but after PM_{2.5} exposure, Phosoho1 was greatly downregulated by Pc (FIG. 4C). Meanwhile, the expression of the key enzyme regulating oxidative phosphorylation, PGC-1α, was repressed by PM_{2.5} exposure but rescued by Pc (FIG. 4D). Taken together, these results suggested that administration of Pc indeed switched the energy metabolism of PM_{2.5}-exposed lung cells to a fatty acid-dependent manner. The utilization of a new energy source alleviated PM_{2.5}-induced cellular dysfunction, which might benefit the growth of the cells.

### Example 4

### Combined Toxicity Analysis

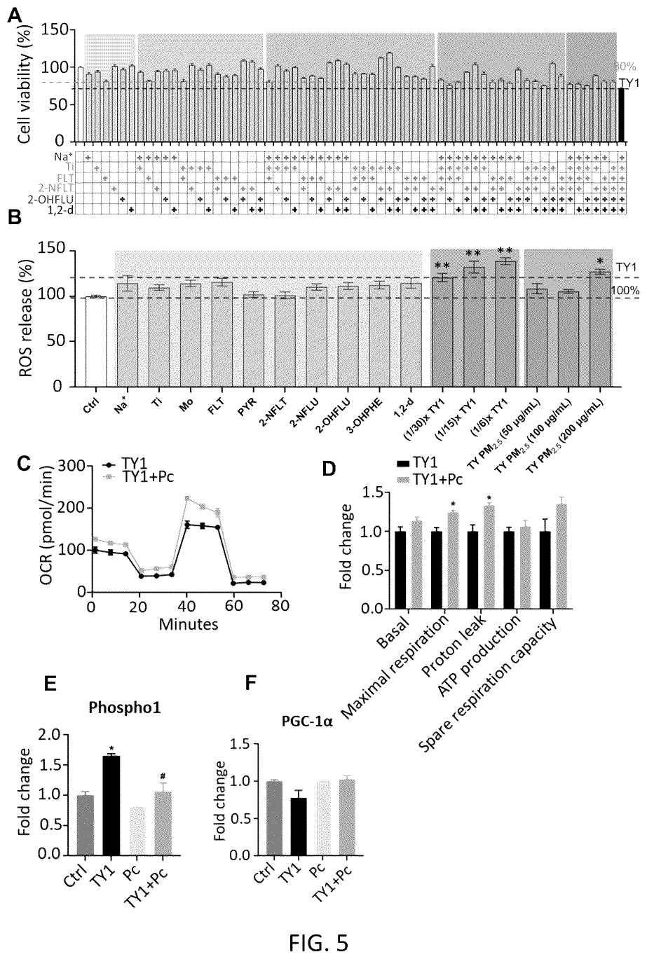

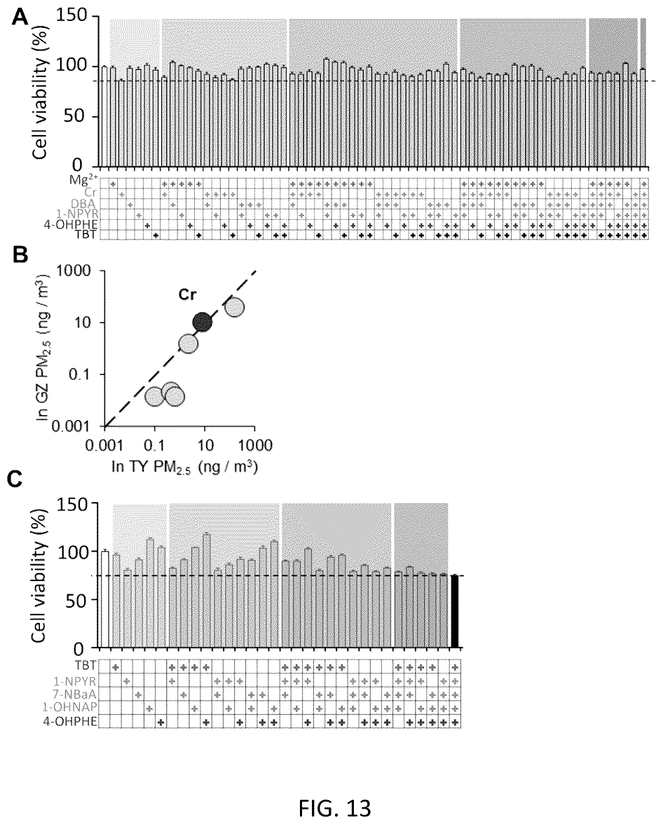

As discussed above, the oxidative potential and toxicities of the PM_{2.5} samples were not solely reliant on PM_{2.5} concentrations, while their compositions, especially in Taiyuan, played a crucial role (FIGS. 1 and 2). The toxicities of the components depend not only on the concentrations of its components but also on their toxic equivalents. To assess the contributions of environmental markers on PM_{2.5} toxicities, the inventors explored the PM_{2.5} components associated with the metabolic changes. Based on the numbers of correlated metabolites and the concentration of each PM_{2.5} component, 6 environmental markers were selected from Taiyuan PM_{2.5}, named panel TY1. According to their average concentrations in winter, the inventors arranged Na⁺ (30 mM), Ti (50 μM), FLT (25 μM), 2-NFLT (1 μM), 2-OHFLU (5 μM) and 1,2-d (0.5 μM) for the toxicity analysis. All 64 combinations were tested, each of which consisted of one to six pollutants in TY1. The mixture of all six contaminants dwindled cell viability to 72% (FIG. 5A), marginally higher than the average cell viability caused by winter samples from Taiyuan (64%, FIG. 7J). The cytotoxicities of these compounds varied, and certain combinations of four or five compounds could decrease cell viability remarkably (<80%). A similar selection in the Guangzhou samples resulted in the panel GZ1 of six different chemicals (Mg²⁺ (500 μM), Cr (50 μM), DBA (1 μM), 1-NPYR (0.05 μM), 4-OHPHE (0.05 μM) and TBT (0.02 μM)), which was less toxic than the panel TY1 (FIG. 13A). Sulfate and nitrate, despite being the most abundant components, failed to meet the selection criteria and were therefore excluded from both panels. This consistently indicates that concentration alone is not enough for dictating the effects of PM_{2.5}. Regarding metals, similarly, Ti and Cr, chosen for the TY1 and GZ1 panels respectively, were not identified as metals with high oxidative potential (FIG. 9). This suggests that some of them may impact cell health through mechanisms other than ROS production. For instance, it appeared that the toxicity of GZ1 mainly came from the high levels of Cr and the mixtures containing Cr exhibited toxicity no higher than Cr (FIG. 13A). Although more sophisticated synergies might exist, this phenomenon could be, at least partially, attributed to the low concentrations of organic components in Guangzhou compared with those in Taiyuan. In panel GZ1, Cr stood out as the only chemical with a higher annual average concentration in Guangzhou (10.9 ng/m³) compared to Taiyuan (7.2 ng/m³), as depicted in FIG. 13B. Other components identified from the Guangzhou PM_{2.5} samples exhibited higher concentrations in Taiyuan instead. To test this possibility, the combinations of chemicals from panel GZ2, 1-NPYR, 7-NBaA, 1-OHNAP, 4-OHPHE and TBT at 1,000 times higher concentrations were used to challenge cells. The results showed that this combination exerted significant toxicity (75%) at a higher concentration (FIG. 13C). The overall results of the combined toxicity analysis suggested that the compounds in organic components played important roles in causing the toxicity of PM_{2.5}. Similarly, a single environmental marker in Taiyuan did not produce ROS, but the mixture of all six at a concentration equivalent to 200 μg/mL PM_{2.5} induced ROS release in cells in a dose-dependent manner (FIG. 5B).

Furthermore, supplying Pc also showed protective effects against the adverse impacts of the TY1 panel on mitochondrial functions (FIGS. 5C and 5D), similar to the results from Taiyuan PM_{2.5} samples. Also, replacing Taiyuan PM_{2.5} samples with the mixture of TY1 resulted in the same expression pattern of Phospho1 and PGC-1α (FIGS. 5E and 5F). Taken together, these results demonstrated that panel TY1 has cytotoxicity on the lung cells similar to the Taiyuan PM_{2.5}, which can be alleviated by an additional supply of Pc.

Based on the above, the inventors determined the major components of whole-year PM_{2.5} collected from Taiyuan and Guangzhou and evaluated the contamination levels, health risks, and cytotoxicities of PM_{2.5} in these two cities. Also, functional metabolomics was conducted to explore differential metabolites after being exposed to Taiyuan or Guangzhou PM_{2.5}. Potential environmental markers and functional metabolites were screened by Pearson's correlation analysis. The results suggested the environmental markers were city-specific and the selected chemical mixture could interact with each other to exhibit combined effects. More importantly, the small molecule Pc has surprising effect on rescuing PM_{2.5}-induced mitochondrial dysfunction via activating the fatty acid oxidation pathway.

### Materials and Methods

### Chemical and Materials

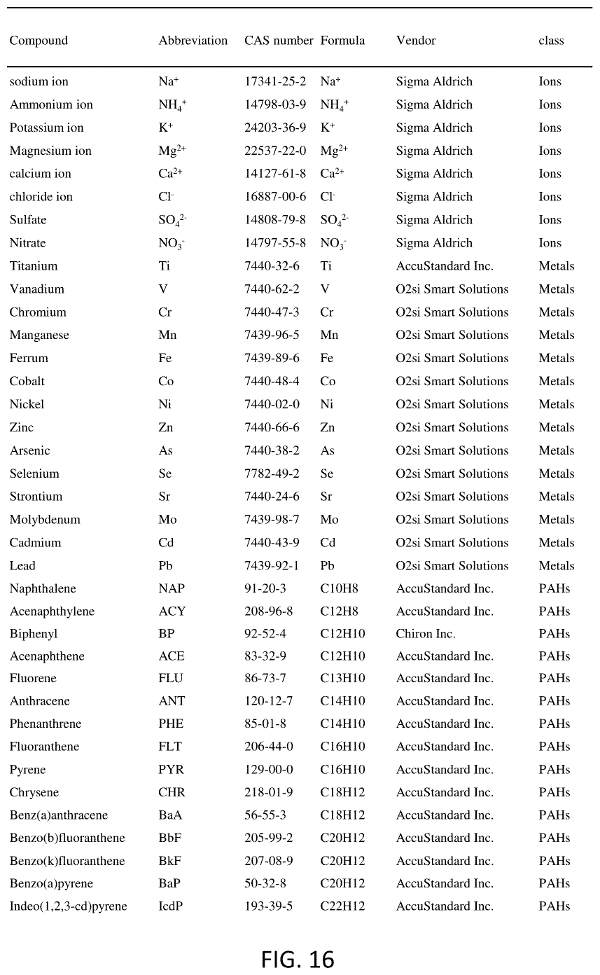

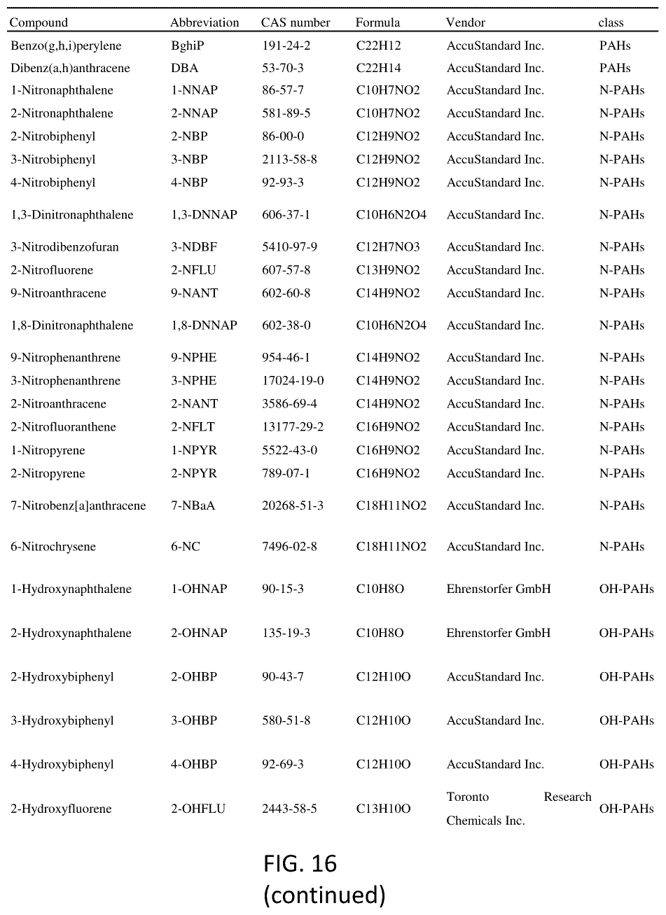

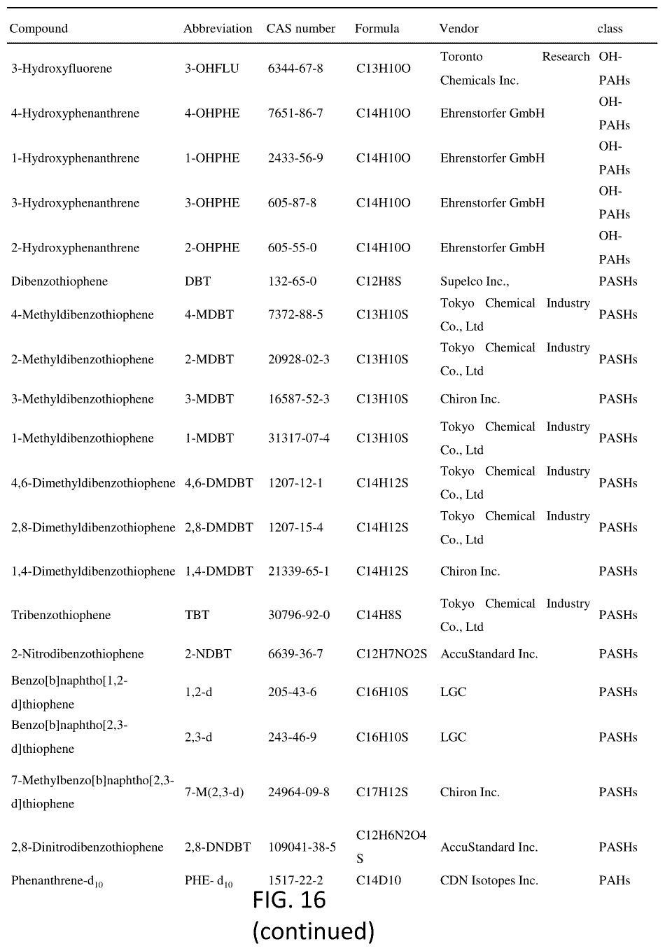

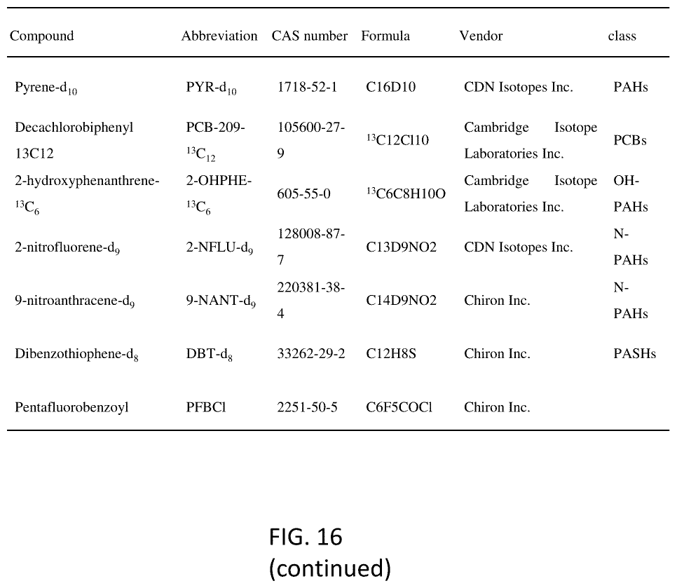

All the reagents and solvents used in the sample analysis were of high-performance liquid chromatography (HPLC) grade or higher. The abbreviations, structures and suppliers of all these compounds are shown in FIG. 16.

### PM_{2.5} Sampling

From Taiyuan and Guangzhou, daily 23.5-h PM_{2.5} samples were collected on quartz microfiber filters (90 mm diameter, Whatman) using a medium volume air sampler (ADS-2062E, AMAE Co., Ltd, Shenzhen, China) at a flow rate of 0.1 m³ min⁻¹. All filters were pre-conditioned at 550° C. for 5 hours before sample collection. After the collection of PM_{2.5}, filters were wrapped with aluminum foil, sealed in a zipped bag, and kept at −80° C. A total of 48 PM_{2.5} samples were collected at each site from May 2017 to April 2018. After excluding the potentially unreliable samples, 45 samples from Taiyuan and 44 samples from Guangzhou were finally used in this study.

### Chemical Analysis

EC and OC were analyzed using a thermal and optical transmittance aerosol carbon analyzer (Sunset Laboratory, Tigard, OR, USA). Metals were quantified by inductively coupled plasma mass spectrometry (ICP-MS, Agilent 7900). Water-soluble inorganic ions were determined by ion chromatography (Thermo Fisher, ICS-5000+, USA). The analysis of PAHs, N-PAHs, OH-PAHs and PASHs were performed according to the methods described in Y. Zhang et al, Chemosphere 198,303-310 (2018), Y. Zhang et al, Talanta 195, 757-763 (2019), and Y. Zhang et al, Chin. Chem. Lett. 32, 801-804 (2021).

### Cell Culture and Exposure

BEAS-2B (Conservation Genetics CAS Kun-ming Cell Bank, Chinese Academy Sciences), a widely used lung cell line, was cultured in BEGM™ bronchial epithelial cell growth medium (CC-3170, Lonza) and maintained in a humidified incubator with 5% CO2 at 37° C. For PM_{2.5} exposure, each one-eighth filter with PM_{2.5} samples was sonicated in 50 mL Milli-Q water for 30 min 5 times at 4° C. The supernatants were freeze-dried and redissolved in 1 mL potassium phosphate buffer (PBS) (Thermo Fisher, USA). Of each sample solution, 100 μL was diluted with growth mediums and added to BEAS-2B cells seeded in each well of 96-well plates. After 24-h exposure, cell viability and lactate dehydrogenase (LDH) release were detected by MTS assay (Promega, USA) and LDH cytotoxicity detection kit (Beyotime, Beijing) with six replicates (n=6).

### DTT Assay

The redox activity of PM_{2.5} and specific components were assessed by DTT assay. Equal volumes of PM_{2.5} solutions were incubated with DTT (100 μM) in 0.1 M PBS at pH 7.4 for 90 min. Then 1 mL of 10% trichloroacetic acid (TCA) was added to the solution and a 0.5 ml aliquot of the mixture was mixed with 1 mL 0.4 M Tris-HCl, pH 8.9 containing 20 mM Ethylenediaminetetraacetic acid (EDTA) and 25 μL of 10 mM 5,5′-Dithiobis-2-nitrobenzoic acid (DTNB). The assay was read at 412 nm.

### Metabolites Extraction and Instrument Analysis

After BEAS-2B cells were grown to around 80% confluency in 6-well plates, every mixture of 100 μL of each sample solution and 900 μl cell medium was applied to a well for 24-h exposure. Cells grown in the standard medium were set as control (n=36). Metabolite extraction was performed. Briefly, cells were extracted with 400 μl ice-cold methanol: water (4:1, v/v). After five times of freeze-thaw using liquid nitrogen followed by a 1-min vortex, the samples were centrifugated for 15 min at a speed of 20,000 g at 4° C. The supernatant was collected and dried in a SpeedVac vacuum concentrator. The dried extracts were then reconstituted in an appropriate volume of methanol: water (1:1, v/v) according to the cell numbers with the lowest volume of 50 μL. After removing insoluble debris with centrifugation, samples were analyzed using an Ultimate 3000 ultra-high performance liquid chromatography (Thermo Scientific, USA) coupled with a Q-Exactive Focus Orbitrap mass spectrometer (Thermo Scientific, USA).

### Metabolite Identification and Correlation Analysis

The metabolite profiling data were extracted by XCMS packages implemented in R as described. After alignment, the significant features were screened with p<0.05 and | fold change |>2 and identified by Compound Discoverer (Thermo Scientific, version 2.1). The PLS-DA plots and heatmaps were conducted using Metaboanalyst. Pearson correlation analysis between pollutant components and metabolites was conducted using SPSS statistics (IBM Company, Chicago, IL, USA).

### Evaluation of the Functions of Screened Metabolites

To detect the cell viability, different concentrations (25 μM, 50 μM, 100 μM, and 200 μM) of selected metabolic markers were added to BEAS-2B cell cultures for 24 h, followed by 200 μg/mL PM_{2.5} samples for another 24 h. After exposure, 10 μL CCK-8 with 100 μL mediums were added to the cells and incubated for 1.5 h, the absorption was detected at 450 nm to assess cell viability. Agilent Seahorse assays were used to evaluate the effects of metabolic markers on mitochondrial functions. The OCR of cells was determined after being treated by both metabolic markers (200 μM) and PM_{2.5} samples (200 μg/mL) (n=3).

### RNA Extraction and Quantitative Real-Time PCR

Total RNA was extracted from BEAS-2B cells with RNAiso plus kit (TaKaRa, Shiga, Japan) according to the manufacturer's instructions. Reverse transcription of cDNA synthesis and real-time quantitative polymerase chain reaction (qPCR) were performed as described previously. All primers synthesized at BGI Co., Ltd. (Hong Kong, China) were described in Table 2.

TABLE 2
Primers applied in q-PCR analysis

| Gene name | Primer sequence (5′→3′) |
|---|---|
| PGC-1α | CACTTACAAGCCAAACCAACAAC |
| | (SEQ ID NO. 1) |
| | CAATAGTCTTGTTCTCAAATGG |
| | GGA (SEQ ID NO. 2) |
| GFAT | CGGCTGCCTGATTTGATT |
| | (SEQ ID NO. 3) |
| | GATAGCCTCGTCCCATTA |
| | (SEQ ID NO. 4) |
| OGT | CAGCATCCCAGCTCACTT |
| | (SEQ ID NO. 5) |
| | CAGCTTCACAGCTATGTCTTC |
| | (SEQ ID NO. 6) |
| Phospho1 | CTTCGACGAGACTATCGTGGA |
| | (SEQ ID NO. 7) |
| | GCCCAGGTACTTGAAGACGC |
| | (SEQ ID NO. 8) |
| 18s | GTAACCCGTTGAACCCCATT |
| | (SEQ ID NO. 9) |
| | CCATCCAATCGGTAGTAGCG |
| | (SEQ ID NO. 10) |

### Combined Toxicity Assessment

Chemical concentrations were calculated by the average of detected values from November 2017 to January 2018. To amplify the biological effects of the chemical mixture, 30 times the calculated concentrations of selected chemicals in Taiyuan and Guangzhou were applied for cell viability assay. For the selected chemicals from PM_{2.5} samples, 1×, 2×, and 5× concentrations equivalent to 200 μg/mL PM_{2.5} were used for ROS production analysis.

### Statistical Analysis

Statistical analysis was conducted with GraphPad Prism software and Microsoft Excel. Data are presented as mean±SEM and p<0.05 was considered to be significant.

### Chemical Analysis

Ultrasonic extraction was used to extract PM_{2.5}-bound PAHs, N-PAHs, OH-PAHs and PASHs. An eighth of each PM_{2.5} sample filter was cut into strips and put into a 150 ml beaker. Then surrogate standards (PHE-d₁₀, 2-OHPHE-¹³C₆ and 2-NFLU-d₉, DBT-d₈) were added for quality control. For PAHs, N-PAHs and OH-PAHs, 50 mL hexane/acetone (1:1, v/v) was used to do ultrasonic extraction of PM_{2.5} twice. A total of 100 ml extract was concentrated into 8 mL and divided into two aliquots. The first part was purified by a silica gel column (27 cm×1 cm i.d.). After activating silica gel and loading samples, 15 mL n-hexane was added to elute impurities, and the analytes were then eluted by 50 ml dichloromethane (DCM). PAHs and NPAHs existed in DCM. Then, 50 mL DCM was concentrated into 1 mL. After spiking with internal standards, PAHs and N-PAHs would be analyzed by APGC-MS/MS. The second aliquot was applied for the determination of OH-PAHs in PM_{2.5}. The extract was dried by nitrogen purge. Then, 0.5 M sodium hydroxide solution (NaOH) (1 mL) was added and washed with 5 mL n-hexane 3 times (15 mL in total). After that, 10% PFBCI solution (300 μL in toluene) was added to do derivatization of phenolic groups in OH-PAHs. Shake for 5 min and react at room temperature for 10 min. After the derivatization, extract the derivatized OH-PAHs with 2 ml n-hexane 5 times (10 mL in total). The extract was concentrated into 1 mL and spiked with PCB-209-¹³C₁₂ as the internal standard. Then it was analyzed by APGC-MS/MS. For PASHs, pure DCM (50 mL) was placed in the beaker for 60 min ultrasonic extraction twice (100 mL/120 min). The DCM extract was concentrated by rotary evaporation from 100 mL to near dryness.

The concentrated sample extract was loaded onto a silica gel column chromatography (27 cm×1 cm i.d.) for purification. After sample loading, PASHs were eluted with 40 ml of hexane/DCM (1:4, v/v). The eluent was then concentrated to 1 ml and submitted to instrumental analysis.

Using Agilent 7890B gas chromatography (Agilent Technologies Inc., USA) equipped with Xevo TQ-S triple-quadrupole mass spectrometer (Waters Corporation., UK), 17 PAHs, 18 N-PAHs, 11 OH-PAHs and 14 PASHs were analyzed, operating in APCI positive mode by three independent injections.

### Metabolomic Analysis

An Ultimate 3000 UHPLC system coupled with a Q-Exactive Focus Orbitrap mass spectrometer (Thermo Scientific) was employed for untargeted metabolomics analysis. Metabolite separation was performed on an Acquity BEH amide column (150×2.1 mm; 1.7 mm) equipped with a guard column (5×2.1 mm; 1.7 μm). In positive ion mode, the mobile phase consisted of water (A) and 95% acetonitrile (B), each containing 10 mM ammonium formate and 0.125% formic acid. In negative ion mode, the mobile phase was composed of water (A) and 95% acetonitrile (B), each with 10 mM ammonium acetate and 0.04% ammonium hydroxide. The elution gradient was set as follows: 0-2 min, 100% B; 2-7.7 min, 100% B-70% B; 7.7-9.5 min, 70% B-40% B; 9.5-10.3 min, 40% B-30% B; 10.3-12.3 min, 30% B; 12.3-14.8 min, 30% B-100% B; 14.8-20 min, 100% B. The mobile phase flow rate was 0.3 mL/min and the injection volume was 5 μL. The column temperature was set as 40° C. The ESI conditions were optimized as follows: capillary voltage, 3.6 kV and −2.6 kV in positive and negative ion mode, respectively; capillary temperature, 350° C.; probe heater temperature, 320° C.; sheath gas flow, 40 arbitrary units; aux gas flow, 10 arbitrary units and sweep gas flow, 1 arbitrary unit. The mass spectrometer was performed in high-resolution full scan mode (mass resolution, 35000). followed by MS/MS scans of the three most abundant ions using DDA.

### Seahorse Analysis

Cell Mito stress test: BEAS-2B cells were seeded in the XF 96-well Seahorse assay miniplates with a density of 20,000 cells/well in BEGM™ bronchial epithelial cell growth medium and incubated for 24 h at 37° C. After exposure, the cells were washed with XF assay medium including 10 mM glucose, 1 mM sodium pyruvate, and 2 mM L-glutamine (adjusted pH to 7.4 with 0.1 M NaOH) and incubated for 1 h in a 37° C. non-CO₂ incubator. Before assay, the sensor cartridge was hydrated in Agilent Seahorse XF Calibrant at 37° C. in a non-CO₂ incubator overnight, and oligomycin (port A final 1 μM), carbonyl cyanide-4-(trifluoromethoxy) phenylhydrazone (FCCP) (port B final 1 μM chosen from the optimization assay) and rotenone antimycin A (port C final 0.5 μM) were added to the corresponding ports of the hydrated sensor cartridge. Then the cartridge was transferred to the Seahorse analyzer (Agilent Technologies) to calibrate the period for approximately 20 min. When the calibration has been completed, the utility plate was removed and the cell miniplate was placed to start the assay. The assay was divided into four parts. After the equilibration period was three basal assays, and then oligomycin was added to inhibit the complex V (ATP synthase) for three cycles, FCCP was followed to stimulate maximal respiration in mitochondria by uncoupling ATP synthesis from electron transport. Lastly, rotenone plus antimycin A was added to determine the non-mitochondrial respiratory rate. Oxygen consumption rate (OCR) was used to assess the mitochondrial respiratory change.

The substrate oxidation stress test was used to assess the specific mitochondrial substrates that were relevant or required for cellular phenotype and function. Two different inhibitors (UK50099 for the glucose pathway and etomoxir for the long-chain fatty acids pathway) were added before the Mito stress test and the following process was the same with the Mito stress test.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A method for treating, alleviating or preventing a disease or condition caused by an air pollutant, comprising administering a composition comprising an effective amount of Pc, or a prodrug thereof, to a subject in need thereof.

2. The method of claim 1, wherein the air pollutant comprises particulate matter.

3. The method of claim 2, wherein the particulate matter has an average aerodynamic diameter of less than 10 μm.

4. The method of claim 2, wherein the particulate matter has an average aerodynamic diameter of less than 2.5 μm.

5. The method of claim 2, wherein the particulate matter comprises predominantly of inorganic ions.

6. The method of claim 1, wherein the subject suffers from or is at risk of developing energy metabolism disorder.

7. The method of claim 1, wherein the disease or disorder is associated with mitochondrial dysfunction in lung cells.

8. The method of claim 1, wherein the Pc activates fatty acid oxidation to mitigate energy deficiencies caused by the air pollutant.

9. The method of claim 1, wherein the composition further comprises taurine, creatine or a combination thereof.

10. The method of claim 1, wherein the composition further comprises UDP-GlcNAc, NAA, or a combination thereof.

11. The method of claim 1, further comprising a step of measuring the level of Pc in a biological sample of the subject to determine if the subject has reduced Pc level compared to an individual unexposed to the air pollutant.

12. A method for improving energy supply in lung cells exposed to an air pollutant, comprising contacting the lung cells with an effective amount of Pc or a prodrug thereof.

13. The method of claim 12, wherein the lung cells have mitochondrial dysfunction.

14. The method of claim 12, wherein the air pollutant comprises particulate matter.

15. The method of claim 14, wherein the particulate matter has an average aerodynamic diameter of less than 2.5 μm.

16. The method of claim 12, wherein the Pc or a prodrug thereof activates fatty acid oxidation to mitigate energy deficiencies caused by the air pollutant.

17. A composition for improving energy supply in lung cells exposed to an air pollutant, the composition comprising an effective amount of Pc or a prodrug thereof for activating fatty acid oxidation in lung cells.

18. The composition of claim 17, wherein the composition further comprises an effective amount of taurine, creatine, UDP-GlcNAc, NAA, or a combination thereof.

19. The composition of claim 17, where the composition comprises both Pc and UDP-GlcNAc.

20. The composition of claim 17, wherein the composition is provided in a form of food supplement.


SEQUENCE LISTING (10 sequences)

SEQ ID NO: 1 - DNA, 23 bp, synthetic construct
  source 1..23: mol_type = other DNA
cacttacaagccaaaccaacaac

SEQ ID NO: 2 - DNA, 25 bp, synthetic construct
  source 1..25: mol_type = other DNA
caatagtcttgttctcaaatgggga

SEQ ID NO: 3 - DNA, 18 bp, synthetic construct
  source 1..18: mol_type = other DNA
cggctgcctgatttgatt

SEQ ID NO: 4 - DNA, 18 bp, synthetic construct
  source 1..18: mol_type = other DNA
gatagcctcgtcccatta

SEQ ID NO: 5 - DNA, 18 bp, synthetic construct
  source 1..18: mol_type = other DNA
cagcatcccagctcactt

SEQ ID NO: 6 - DNA, 21 bp, synthetic construct
  source 1..21: mol_type = other DNA
cagcttcacagctatgtcttc

SEQ ID NO: 7 - DNA, 21 bp, synthetic construct
  source 1..21: mol_type = other DNA
cttcgacgagactatcgtgga

SEQ ID NO: 8 - DNA, 20 bp, synthetic construct
  source 1..20: mol_type = other DNA
gcccaggtacttgaagacgc

SEQ ID NO: 9 - DNA, 20 bp, synthetic construct
  source 1..20: mol_type = other DNA
gtaacccgttgaaccccatt

SEQ ID NO: 10 - DNA, 20 bp, synthetic construct
  source 1..20: mol_type = other DNA
ccatccaatcggtagtagcg